(19) United States
(12) Patent Application Publication
(10) Pub. No.: US 2023/0212686 A1
(43) Pub. Date: Jul. 6, 2023

(54) KIT AND METHOD FOR DETECTING TARGET NUCLEIC ACIDS USING MAGNETIC NANOPARTICLES

(71) Applicants: Korea University Research and Business Foundation, Seoul (KR); Industry-University Cooperation Foundation Hanyang University ERICA Campus, Ansan-si (KR)

(72) Inventors: Young Keun KIM, Seoul (KR); Ju Hun LEE, Seoul (KR); Jeong Ook SOH, Seoul (KR); Hyeon Su PARK, Seoul (KR); Bum Chul PARK, Seoul (KR)

(73) Assignees: Korea University Research and Business Foundation, Seoul (KR); Industry-University Cooperation Foundation Hanyang University ERICA Campus, Ansan-si (KR)

(21) Appl. No.: 17/980,974

(22) Filed: Nov. 4, 2022

(30) Foreign Application Priority Data
Dec. 31, 2021 (KR) 10-2021-0194433
Jul. 29, 2022 (KR) 10-2022-0094665

(51) Int. Cl. C12Q 1/6886; B01L 3/00

(52) U.S. Cl. CPC C12Q 1/6886; B01L 3/502761; B01L 2200/0668; B01L 2300/0645; B01L 2200/16; C12Q 1/6844

(57) ABSTRACT

Provided are a kit and a method for detecting target nucleic acids using magnetic nanoparticles. The kit for detecting target nucleic acids includes a reactor having an opening on one side and provided with a sample containing target nucleic acids, at least one magnetic nanoparticle part provided in the reactor, a conductive substrate provided to cover the opening of the reactor, and a magnetic field applying part for applying a magnetic field to the reactor, in which the magnetic nanoparticle part includes a magnetic nanoparticle including a core portion made of iron oxide and a shell portion made of gold and provided to surround the core portion, and a primer attached to the shell portion of the magnetic nanoparticle.

### CROSS-REFERENCE TO RELATED APPLICATIONS

This application claims priority to and the benefit of Korean Patent Application Nos. 10-2021-0194433 filed on Dec. 31, 2021 and 10-2022-0094665 filed on Jul. 29, 2022, in the Korean Intellectual Property Office, the entire contents of which are incorporated herein by reference.

### TECHNICAL FIELD

The present disclosure relates to a kit and a method for detecting target nucleic acids using magnetic nanoparticles, and more particularly, to a kit and a method for detecting target nucleic acids using magnetic nanoparticles capable of effectively detecting target nucleic acids at a low concentration in a short time with high sensitivity.

### BACKGROUND ART

Molecular diagnosis diagnoses a basis of disease such as DNA or RNA, and has been used in various fields, such as infectious diseases, cancer diagnosis, genetic diseases, and customized diagnosis. As a representative molecular diagnostic technology, there is a polymerase chain reaction (PCR) technology that amplifies DNA within a short time.

A PCR-based molecular diagnostic technology has advantages for specific cancers and specific viral infectious diseases with high diagnostic accuracy and thus has been used as a gold standard. Among them, real-time PCR, which is expensive, but has the highest accuracy, is mainly used, and a lot of cheaper conventional PCRs have been used.

On the other hand, such a PCR technology has disadvantages of inducing sufficient polymerase chain reaction and requiring expensive equipment, costs, and a lot of time, in order to maintain high accuracy. In addition, there are many difficulties in detecting a base sequence with a short lifespan and a low concentration (1 fM or less).

### SUMMARY OF THE INVENTION

The present disclosure has been made in an effort to provide a kit and a method for detecting target nucleic acids using magnetic nanoparticles capable of detecting a low concentration of target nucleic acids in a short time with high efficiency.

The present disclosure has also been made in an effort to provide a kit and a method for detecting target nucleic acids using magnetic nanoparticles capable of reducing the cycle number of PCR to dramatically shorten the time required for diagnosis and increase efficiency by using novel magnetic nanoparticles.

According to an aspect of the present disclosure, embodiments of present disclosure include a kit for detecting target nucleic acids using magnetic nanoparticles and a method for detecting target nucleic acids.

An embodiment of the present disclosure provides a kit for detecting target nucleic acids including a reactor having an opening on one side, at least one magnetic nanoparticle part provided in the reactor, a conductive substrate provided to cover the opening of the reactor, and a magnetic field applying part for applying a magnetic field to the reactor, in which the magnetic nanoparticle part includes a magnetic nanoparticle including a core portion made of iron oxide and a shell portion made of gold and provided to surround the core portion, and a primer attached to the shell portion of the magnetic nanoparticle.

In an embodiment, an average diameter of the magnetic nanoparticle may be provided with a first length and an average thickness of the shell portion may be provided with a second length, in which the second length may be 0.09 to 0.15 times greater than the first length.

In an embodiment, an average diameter of the magnetic nanoparticle may be provided with a first length and an average thickness of the shell portion may be provided with a second length, in which the first length may be 170 nm to 300 nm and the second length may be 15 nm to 40 nm.

In an embodiment, the magnetic nanoparticle part may be prepared by preparing a core portion made of iron oxide, providing a buffer portion containing silicon on the surface of the core portion, preparing magnetic nanoparticles by functionalizing an outer surface of the buffer portion with at least one of an amino group (—NH₂) and a thiol group (—SH) and forming a shell portion containing gold thereon, and mixing the magnetic nanoparticles and a thiolated reverse primer.

In an embodiment, the primer may include a reverse primer or a forward primer, and the kit may further include an electrochemical signal measuring part for measuring an electrochemical signal using a first electrode, a second electrode, and a three-electrode module including the first electrode, the second electrode, and a conductive substrate, which are provided in the reactor.

In an embodiment, the first electrode may include a reference electrode, the conductive substrate may include a working electrode, and the second electrode may include a counter electrode controlling an electron balance generated from the reference electrode and the working electrode, which are performed by a three-electrode module, in which the first or second electrode may consist of any one or more of gold (Au), cobalt (Co), platinum (Pt), silver (Ag), carbon nanotube, graphene, and carbon, and the conductive substrate may include any one or more of indium tin oxide (ITO), ZnO, SnO₂, In₂O₃, CdSnO₄, a carbon substrate material including carbon nanotubes, a fluorine-doped tin oxide (FTO) added with fluorine, and an aluminum doped zinc oxide (AZO) added with aluminum.

In an embodiment, the electrochemical signal measuring part may include any one or more of a differential pulse voltammeter (DPV), an anodic stripping voltammetry (ASV), a chronoamperometry (CA), a cyclic voltammetry, a square wave voltammetry (SWV), and an impedance meter.

In an embodiment, the magnetic field applying part may reversibly apply a magnetic field into the reactor one or more times with the conductive substrate interposed therebetween. The magnetic field applying part may move the magnetic nanoparticle part toward the opening of the reactor and control the degree of moving the magnetic nanoparticle part.

In an embodiment, the sample containing the target nucleic acids may include any one or more of blood, serum, plasma, saliva, ascites, amniotic fluid, semen, lacrimal fluid, cerebrospinal fluid, bone marrow, pleural fluid, synovial fluid, lymph, urine, tissue biopsies and cell lines.

In an embodiment, the target nucleic acid may include cell free DNA (cfDNA) or circulating tumor DNA (ctDNA).

According to another aspect of the present disclosure, there is provided a method for detecting target nucleic acids using magnetic nanoparticles including preparing a magnetic nanoparticle part; obtaining a magnetic nanoparticle part with an amplified product collected on the surface by adding the magnetic nanoparticle part in a sample including target nucleic acids to perform polymerase chain reaction (PCR); adding the magnetic nanoparticle part with the amplified product collected on the surface in the reactor provided with an opening, covering the opening with a conductive substrate, and applying a magnetic field into the reaction from one side of the conductive substrate under a first condition; adding metal ions including any one or more of ruthenium (Ru), iron (Fe), silver (Ag), copper (Cu), nickel (Ni), cadmium (Cd) and zinc (Zn) into the reactor and then applying the magnetic field under a second condition; and providing first and second electrodes in the reactor, and measuring an electrochemical signal using the first and second electrodes and the conductive substrate together.

In an embodiment, the preparing of the magnetic nanoparticle unit may include preparing a core portion made of iron oxide, providing a buffer portion including silicon on the surface of the core portion to prepare a pre-nanoparticle, preparing a magnetic nanoparticle by functionalizing an outer surface of the buffer portion with at least one of an amino group (—NH₂) and a thiol group (—SH) and forming a shell portion containing gold thereon, and mixing the magnetic nanoparticle and a primer and performing salt aging.

In an embodiment, an average diameter of the magnetic nanoparticle may be provided with a first length and an average thickness of the shell portion may be provided with a second length, in which the first length may be 170 nm to 300 nm and the second length may be 15 nm to 40 nm.

In an embodiment, the preparing of the magnetic nanoparticle may include impregnating the pre-nanoparticles in a solution containing an amine group and then sonicating the pre-nanoparticles at a temperature of 60° C. to 90° C. for 2 hours to 10 hours, washing the pre-nanoparticles using water after the sonication is completed and dispersing the washed pre-nanoparticles in water to prepare a pre-nanoparticle dispersion, and forming a shell portion by mixing gold nanoparticles and then adding a chloroauric acid (HAuCl₄) solution to prepare the pre-nanoparticle dispersion as magnetic nanoparticles, in which the gold nanoparticles may be prepared in advance in the form of particles having an average diameter of 0.5 nm to 5 nm and used as a seed.

In an embodiment, the primer may include a single thiolated reverse primer or forward primer, and the salt aging may include adding and mixing the magnetic nanoparticles and the primers to a stirrer, and then adding a first solution and a second solution in a volume ratio of 5:1 to 30:1 in the stirrer a plurality of times and sonicating the mixture, in which the first solution may be provided by mixing a sodium chloride solution, a phosphate buffered saline, and ultrapure water, and the second solution may be provided by mixing a phosphate buffered saline, sodium dodecyl sulfate and ultrapure water.

In an embodiment, by applying the magnetic field under the first and second conditions, at least a part of the magnetic nanoparticle part may be transferred toward the opening of the reactor to be in contact with the conductive substrate, the first condition may include a condition of 0.1 T to 1 T for 1 minute to 10 minutes, and the second condition may include a condition of 0.1 T to 1 T for 12 minutes to 30 minutes.

In an embodiment, in the measuring of the electrochemical signal, the first electrode may include a reference electrode, the conductive substrate may include a working electrode, and the second electrode may include a counter electrode controlling an electron balance generated from the reference electrode and the working electrode, which may be performed as a three-electrode module, in which the first or second electrode may consist of any one or more of gold (Au), cobalt (Co), platinum (Pt), silver (Ag), carbon nanotube, graphene, and carbon, and the conductive substrate may include any one or more of indium tin oxide (ITO), ZnO, SnO₂, In₂O₃, CdSnO₄, a carbon substrate material including carbon nanotubes, a fluorine-doped tin oxide (FTO) added with fluorine, and an aluminum doped zinc oxide (AZO) added with aluminum.

In an embodiment, the measuring of the electrochemical signal may include any one or more of a differential pulse voltammeter (DPV), an anodic stripping voltammetry (ASV), a chronoamperometry (CA), a cyclic voltammetry, a square wave voltammetry (SWV), and an impedance meter.

In an embodiment, the first electrode may be a reference electrode containing Ag/AgCl, the second electrode may be a counter electrode containing Pt, and the conductive substrate may be a working electrode containing ITO, which may be performed as a three-electrode module, and the measuring of the electrochemical signal may include a differential pulse voltammetry (DPV).

According to the present disclosure, it is possible to provide a kit and a method for detecting target nucleic acids using magnetic nanoparticles capable of detecting various types of cancers, viruses, and the like with high sensitivity by variously controlling the shape and the like of the magnetic nanoparticles.

Further, it is possible to provide a kit and a method for detecting target nucleic acids using magnetic nanoparticles capable of reducing the cycle number of PCR and detecting diseases quickly with high accuracy.

### BRIEF DESCRIPTION OF THE DRAWINGS

FIG. 1 is a diagram schematically illustrating a kit for detecting target nucleic acids of the present disclosure.

FIG. 2 is a diagram illustrating magnetic nanoparticles of the present disclosure.

FIG. 3 is a diagram schematically illustrating a method of manufacturing a magnetic nanoparticle part according to an embodiment of the present disclosure.

FIG. 4 is a schematic diagram and a TEM image of magnetic nanoparticles of Preparation Example 3.

FIG. 5 is an SEM image of magnetic nanoparticles of Preparation Example 3.

FIG. 6 is a graph showing sizes of the magnetic nanoparticles of Preparation Example 3 and iron oxide nanoparticles (core) according to Preparation Example 1.

FIG. 7 is a result of confirming magnetic field characteristics of the magnetic nanoparticles of Preparation Example 3.

FIG. 8 is a diagram schematically illustrating a comparison before and after performing polymerase chain reaction.

FIG. 9 is a diagram schematically illustrating a device for detecting target nucleic acids using magnetic nanoparticles of the present disclosure and a method of using the same.

FIG. 10 is a diagram schematically illustrating an amplification reaction mechanism using the magnetic nanoparticle part in FIG. 9.

FIG. 11 is a schematic diagram illustrating a difference in current signal between a magnetic nanoparticle having a product with amplified long base pairs and a magnetic nanoparticle having only a reverse primer.

FIG. 12 is a schematic description of a rearrangement of magnetic nanoparticles that cause a difference between an initial current signal and a maximum current signal.

FIG. 13 is a result of confirming current signals according to the DPV measurement number by varying a concentration of target nucleic acids using magnetic nanoparticles (control) attached with only a reverse primer and magnetic nanoparticle having an amplified product, respectively.

FIG. 14 illustrates the results of FIG. 13 by a current amplified ratio (CAR).

FIG. 15 is a result of confirming magnetic nanoparticles (control) attached with only a reverse primer and magnetic nanoparticles having an amplified product by reducing the cycle number of polymerase chain reaction.

FIG. 16 is a result confirmed by varying a ratio of magnetic nanoparticles (control) attached with only a reverse primer and magnetic nanoparticles having an amplified product.

### DETAILED DESCRIPTION

Details of other exemplary embodiments will be included in the detailed description and the accompanying drawings.

Advantages and features of the present disclosure, and methods for accomplishing the same will be more clearly understood from exemplary embodiments to be described below in detail with reference to the accompanying drawings. However, the present disclosure is not limited to embodiments to be disclosed below, but may be implemented in a variety of different forms. Unless otherwise specified in the description below, all numbers, values and/or expressions expressing components, reaction conditions, and contents of components in the present disclosure are approximations which reflect various uncertainties in measurements generated to obtain these values among other things essentially different from these numbers, and thus, in all cases, it is to be understood as being modified by the term “about”. Further, when numerical ranges are disclosed in this description, these ranges are continuous and include all values from a minimum value to a maximum value of the range, unless otherwise indicated. Furthermore, when such ranges refer to integers, all integers including from the minimum to the maximum are included, unless otherwise indicated.

Further, in the present disclosure, when a range is disclosed for variables, the variables will be understood to include all values within the disclosed range, including disclosed endpoints of the range. For example, it will be understood that a range of “5 to 10” includes nay lower range such as 6 to 10, 7 to 10, 6 to 9, and 7 to 9 as well as values 5, 6, 7, 8, 9, and 10, and includes any value between integers that fall within the disclosed range, such as 5.5, 6.5, 7.5, 5.5 to 8.5 and 6.5 to 9, etc. For example, it will be understood that a range of “10% to 30%” includes any lower range such as 10% to 15%, 12% to 18%, and 20% to 30% as well as all integers including values of 10%, 11%, 12%, 13%, etc. and up to 30%, and includes any value between integers that fall within the disclosed range, such as 10.5%, 15.5%, 25.5%, etc.

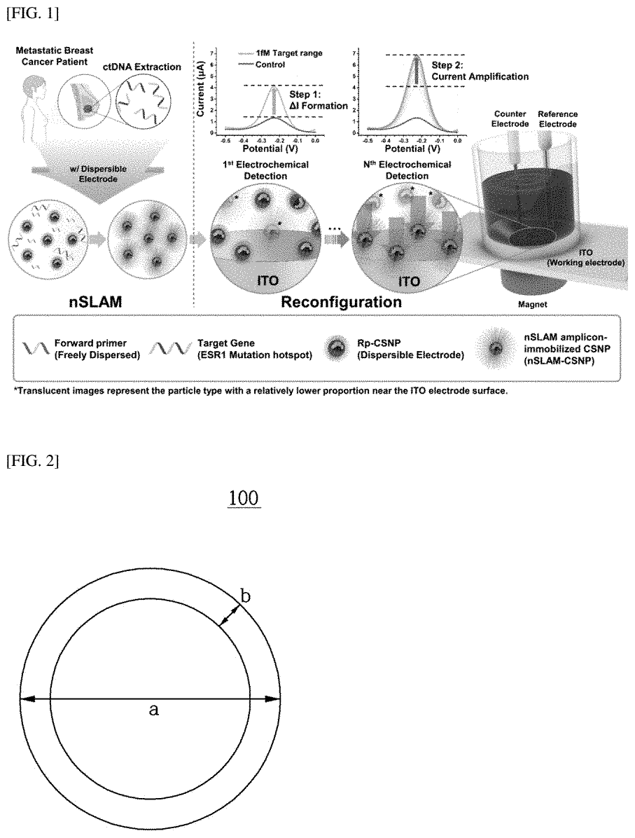

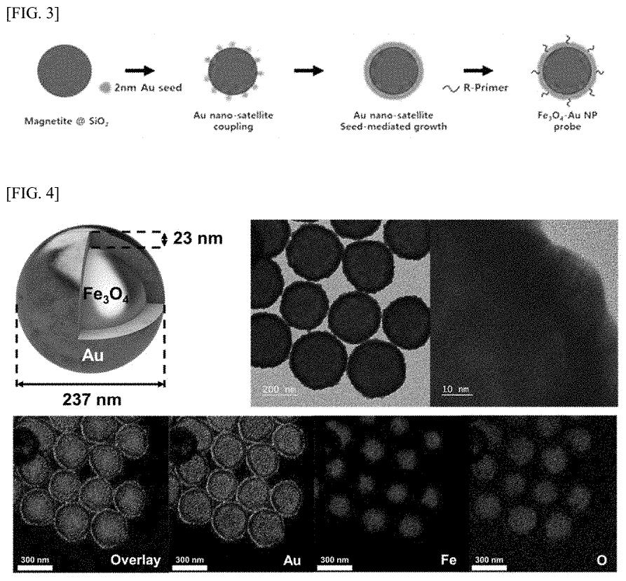

FIG. 1 is a diagram schematically illustrating a kit for detecting target nucleic acids of the present disclosure. FIG. 2 is a diagram illustrating magnetic nanoparticles of the present disclosure. FIG. 3 is a diagram schematically illustrating a method of manufacturing a magnetic nanoparticle part according to an embodiment of the present disclosure.

Referring to FIGS. 1 to 3, a kit for detecting target nucleic acids using magnetic nanoparticles 100 according to an embodiment of the present disclosure includes a reactor having an opening on one side; at least one magnetic nanoparticle part provided in the reactor; a conductive substrate provided to cover the opening of the reactor; and a magnetic field applying part for applying a magnetic field to the reactor. The magnetic nanoparticle part includes a magnetic nanoparticle consisting of a core portion made of iron oxide and a shell portion made of gold provided to surround the core portion, and a primer attached to the shell portion of the magnetic nanoparticle.

In the present disclosure, the terms “target nucleic acid”, “single target base sequence”, and “specific single base sequence” refer to a nucleic acid molecule to be amplified by the method according to the present disclosure. The type of nucleic acid may be deoxyribonucleotide (DNA), ribonucleotide (RNA), and a mixture or combination thereof. Bases constituting the nucleic acid are naturally occurring nucleotides, for example, guanine (G), adenine (A), thymine (T), cytosine (C), and uracil (U), but may contain other natural and artificial modified bases. The term “modified base” refers to a base in which five nucleotides of guanine, adenine, thymine, cytosine, and uracil have been chemically modified. In the present disclosure, the target nucleic acid, the single target base sequence, and the specific single base sequence need to be single-stranded for amplification, but a nucleic acid that forms a double-strand or higher-order structure may also be converted and used into a single chain by heat denaturation, alkali denaturation treatment, etc. The target nucleic acid, the single target base sequence, and the specific single base sequence of the present disclosure also include aspects in which such denaturation treatment is added. In addition, cDNAs prepared by reverse transcription reaction using RNA as a template is included.

As used herein, the term “sample” refers to a mixture considered to include a target nucleic acid to be detected, a single target base sequence, and a specific single base sequence. The sample may be derived from a living body including a human (e.g., blood, saliva, body fluid, body tissue, etc.), an environment (e.g., soil, seawater, environmental water (hot spring water, bath water, cooling tower water, etc.)), or an artificial or natural product (e.g., processed foods such as bread, fermented foods such as yogurt, or cultivated plants such as rice and wheat, microorganisms, viruses) and may generally use those that are subjected to a nucleic acid extraction operation. If necessary, a nucleic acid purification process may be added.

The magnetic nanoparticle part may be provided in the reactor after PCR amplification reaction is performed. The opening, for example, one or more holes, may be provided in a lower side of the reactor, and the opening is blocked by the conductive substrate, so that the magnetic nanoparticle part provided in the reactor is not discharged to the outside. The magnetic field applying part is provided on the outside of the conductive substrate, and magnetic field applying part may move the magnetic nanoparticle part toward the opening of the reactor and controls the degree of movement to improve the nucleic acid detection efficiency.

The magnetic nanoparticle means a particle shape including a core portion made of iron oxide and a shell portion made of gold, and the magnetic nanoparticle part means a shape in which primers are attached onto the surface of the magnetic nanoparticle. In addition, an amplified product refers to a result obtained after performing the polymerase chain reaction, and the amplified product may be performed in the primers of the magnetic nanoparticle part and collected on the surface of the magnetic nanoparticle.

Generally, in a process of detecting nucleic acids and the like using PCR technology, there are disadvantages of inducing sufficient a polymerase chain reaction and requiring expensive equipment, costs, and a lot of time, in order to maintain high accuracy. In addition, there are a lot of difficulties to detect a base sequence in which the life of the amplified product is short, and the concentration contained in the sample is low (approximately, 1 fM or less). Therefore, there is a problem in that a nanostructure including a microelectrode, which is studied to induce the interaction between an electrode that has a direct influence, and a target base sequence has low efficiency.

In addition, in the case of a functional metal nanoparticle-based fluorescence or electrical signal detection technology used together with conventional PCR technology, there is a problem in that after the polymerase chain reaction, a post-treatment process for the particles is involved, and in a process of creating an environment for the particles to react specifically with the target base sequence, the detection efficiency is lowered, and the probability of causing a detection error is high.

In an embodiment of the present disclosure, after the magnetic nanoparticle is synthesized, a base sequence (forward or reverse primer) that serves to initiate an amplification reaction for a single target base sequence related to a specific disease is attached to the surface of the magnetic nanoparticle using a thiol group to be dispersed in an aqueous solution, thereby improving process efficiency. In addition, a current peak that gradually increases at 0.25 V is measured by adsorbing ruthenium ions to the base sequence to provide an ultra-sensitive disease detection system that can be measured even in a concentration z M of the target base sequence and an area in a concentration range below the concentration.

In addition, the kit for detecting the target nucleic acids using the magnetic nanoparticles according to the embodiment has improved accuracy despite remarkably reducing repetitive cycles in the PCR, and may reduce the PCR process to less than 10 minutes, thereby significantly shortening the time taken for the detection step.

The average diameter of the magnetic nanoparticle 100 is provided with a first length a with reference to FIG. 2, the average thickness of the shell portion is provided with a second length b, and the second length b may be provided 0.09 times to 0.15 times the first length a. Specifically, the second length b may be provided about 0.1 times to 0.15 times, or about 0.5 times to 0.15 times, about 0.7 times to 0.15 times, about 0.09 times to 0.15 times, about 0.09 times to 0.12 times the first length a. If the second length b is less than 0.09 times the first length a, it is difficult to form a uniform thickness of the shell portion and it is difficult to efficiently control the primer attached to the shell portion and the PCR amplified product, and if the second length b is more than 0.15 times the first length a, it is difficult to control the movement of particles when a magnetic field is applied, and it is difficult to effectively detect an electrochemical signal.

In addition, when the first length a is 170 nm to 300 nm, the second length b may be 15 nm to 40 nm. When the first length a is less than 170 nm, there is a problem that the magnetic nanoparticles are agglomerated or aggregated in the sample, and when the first length a is more than 300 nm, it is difficult to control the movement of the magnetic nanoparticles by applying the magnetic field. In addition, when the second length b is less than 15 nm, it is difficult to form a shell portion having a uniform thickness, and when the second length b is more than 40 nm, there is a problem that the sensitivity of the movement of the magnetic nanoparticles is deteriorated by a magnetic field or current. Specifically, the first length may be about 200 nm to 300 nm, about 210 nm to 300 nm, about 220 nm to 300 nm, about 200 nm to 280 nm, or about 200 nm to 260 nm. In addition, the second length b may be about 20 nm to 40 nm, about 20 nm to 35 nm, or about 20 nm to 30 nm.

The magnetic nanoparticle part may be prepared by preparing a core portion made of iron oxide, providing a buffer portion containing silicon on the surface of the core portion, functionalizing the outer surface of the buffer portion with an amino group (—NH₂) or a thiol group (—SH), and then forming a shell portion containing gold to manufacture a magnetic nanoparticle, and mixing the magnetic nanoparticle and a thiolated primer.

The core portion may use an iron chloride solution as an iron precursor, and the core portion made of iron oxide may be prepared by a polyol reaction using glycol and the like serving as a reducing agent and a solvent. The shell portion may be prepared by first preparing gold nanoparticles using a chloroauric acid (HAuCl₄) solution as a gold precursor, attaching the gold nanoparticles to the surface of the core portion, and then growing gold. In this case, in order to stably attach the gold nanoparticles, a buffer portion containing silica may be formed on the surface of the core portion using a silica precursor, and then attached to the core portion through the buffer portion.

Subsequently, a primer may be bound to the shell portion, and the primer may include a reverse primer or a forward primer.

As such, in the magnetic nanoparticle part in which the reverse primer or the forward primer is bound to the surface of the magnetic nanoparticle, when the reverse primer or the forward primer and the target nucleic acids are subjected to the PCR reaction together, a base sequence amplified by the length of the target nucleic acids is double-stranded on the surface of the magnetic nanoparticle. Subsequently, after the magnetic nanoparticle part having an amplified product is provided in the reactor by the PCR reaction, a magnetic field may be reversibly applied into the reactor one or more times with the conductive substrate therebetween by the magnetic field applying part. By the application of the magnetic field, the magnetic nanoparticle part may move toward the opening of the reactor, and may be provided adjacent to the conductive substrate through the opening.

The kit may further include an electrochemical signal measuring part for measuring an electrochemical signal using a first electrode, a second electrode, and a three-electrode module including the first electrode, the second electrode, and a conductive substrate, which are provided in the reactor.

The first electrode includes a reference electrode, the conductive substrate includes a working electrode, and the second electrode includes a counter electrode controlling an electron balance generated from the first reference electrode and the working electrode, which may be performed as the three-electrode module.

The first or second electrode may consist of any one or more of gold (Au), cobalt (Co), platinum (Pt), silver (Ag), carbon nanotube, graphene, and carbon, and the conductive substrate may include any one or more of indium tin oxide (ITO), ZnO, SnO₂, In₂O₃, CdSnO₄, a carbon substrate material including carbon nanotubes, a fluorine-doped tin oxide (FTO) added with fluorine, and an aluminum doped zinc oxide (AZO) added with aluminum.

In addition, the electrochemical signal measuring part may measure an electrochemical signal by using the first and second electrodes and the conductive substrate. For example, the electrochemical signal measuring part may include any one or more of a differential pulse voltammeter (DPV), an anodic stripping voltammetry (ASV), a chronoamperometry (CA), a cyclic voltammetry, a square wave voltammetry (SWV), and an impedance meter.

In the embodiment of the present disclosure, the sample containing the target nucleic acids may include any one or more of blood, serum, plasma, saliva, ascites, amniotic fluid, semen, lacrimal fluid, cerebrospinal fluid, bone marrow, pleural fluid, synovial fluid, lymph, urine, tissue biopsies and cell lines. In addition, the target nucleic acid may be cell free DNA (cfDNA) or circulating tumor DNA (ctDNA).

The cfDNA is cell-free DNA, and refers to a fragment of DNA that does not exist in a cell nucleus and floats in the blood. In healthy people, plasma-free DNA is released from stem cells to maintain a low concentration, but it is known that the concentration increases in physiological phenomena or various clinical conditions such as acute trauma, cerebral infarction, exercise, transplantation, infection, or cancer patients.

Due to these characteristics, the plasma-free DNA has been currently demonstrated in non-invasive prenatal testing for testing fetal sex, genetic mutation or chromosomal abnormalities.

The ctDNA is circulating tumor DNA, and refers to a fragment of DNA derived from a tumor that does not exist in cells and floats in the blood. The ctDNA is a broader term to describe DNA that circulates freely in the bloodstream, but needs to be distinguished from cell-free DNA (cfDNA) that is not a tumor origin. Since the ctDNA may reflect a full tumor genome, the ctDNA has had attention in its potential clinical utility as “liquid biopsies” in the form using the blood. In healthy patients, the level of the cfDNA is only present at a low level, but in cancer patients, a high level of ctDNA may be detected. This may occur due to inefficient penetration of immune cells to a tumor site, so that the effective clearance of ctDNA is reduced from the bloodstream.

According to another aspect of the present disclosure, the present disclosure includes a method for detecting target nucleic acids using the kit for detecting the target nucleic acids using the magnetic nanoparticles described above.

The method for detecting the target nucleic acids using the magnetic nanoparticles includes preparing a nanoparticle part; obtaining an amplified product containing a magnetic nanoparticle part by performing polymerase chain reaction (PCR) by adding the magnetic nanoparticle part to a sample containing the target nucleic acids; putting the amplified product into a reactor having an opening, covering the opening with a conductive substrate, and applying a magnetic field into the reactor from one side of the conductive substrate under a first condition; applying a magnetic field under a second condition after adding metal ions containing at least one of ruthenium (Ru), iron (Fe), silver (Ag), copper (Cu), nickel (Ni), cadmium (Cd) and zinc (Zn) into the reactor; and providing first and second electrodes in the reactor, and measuring an electrochemical signal using the first and second electrodes and the conductive substrate together.

The preparing of the magnetic nanoparticle part may include preparing a core portion made of iron oxide, providing a buffer portion containing silicon on the surface of the core portion to prepare pre-nanoparticles, functionalizing the outer surface of the buffer portion with at least one of an amino group (—NH₂) and a thiol group (—SH) and then forming a shell portion containing gold to prepare magnetic nanoparticles, mixing the magnetic nanoparticles and the primers, and performing salt aging.

The magnetic nanoparticle part may be formed by preparing the magnetic nanoparticles and then attaching the primers to the surface of the magnetic nanoparticles.

The preparing of the magnetic nanoparticles is preparing the core portion made of iron oxide by using an iron chloride solution as an iron precursor and a polyol reaction using glycol, etc. serving as a reducing agent and a solvent. Subsequently, the shell portion may be prepared by forming the buffer portion containing silica on the surface of the core portion using a silica precursor, attaching gold nanoparticles as a seed, and then growing gold. Gold nanoparticles may be attached to the surface of the pre-nanoparticles formed from the core portion to the buffer portion, and in this case, the gold nanoparticles may be functionalized using an amine group and then attached.

Specifically, the pre-nanoparticles are impregnated in a solution containing an amine group, sonicated at a temperature of 60° C. to 90° C. for 2 hours to 10 hours, and washed with water after the sonication is completed, and then the washed pre-nanoparticles are dispersed in water to prepare a pre-nanoparticle dispersion. The pre-nanoparticle dispersion may be prepared as magnetic nanoparticles by mixing gold nanoparticles and then adding a chloroauric acid (HAuCl₄) solution to form the shell portion. In this case, the gold nanoparticles may be prepared in advance in the form of particles having an average diameter of 0.5 nm to 5 nm and used as a seed.

The sonication is performed at a temperature of 60° C. to 90° C. for 2 hours to 10 hours, so that the amine group may be uniformly attached to the surface of the pre-nanoparticles, and process efficiency may be improved in a subsequent process of attaching the gold nanoparticles. In addition, the gold nanoparticles used as the seed may have an average diameter of 0.5 nm to 5 nm, and when the average diameter is less than 0.5 nm, the gold nanoparticles are agglomerated or aggregated on a part of the surface of the pre-nanoparticles, and when the average diameter is more than 0.5 nm, it becomes difficult to control the thickness of the shell portion. Specifically, the average diameter of the gold nanoparticles may be about 1 nm to 5 nm, about 0.5 nm to 4 nm, or about 0.5 nm to 3 nm.

The primer may comprise a single thiolated primer. In the primer, since a double thiol (—SH) group is bound in a 5′ direction capable of reacting with the vicinity of 5′ of a specific single base sequence, the double thiol bond may be processed into a single thiol bond. The thiol group (—SH) may modify a reverse primer or a forward primer by using Chemical Formula 1 below.

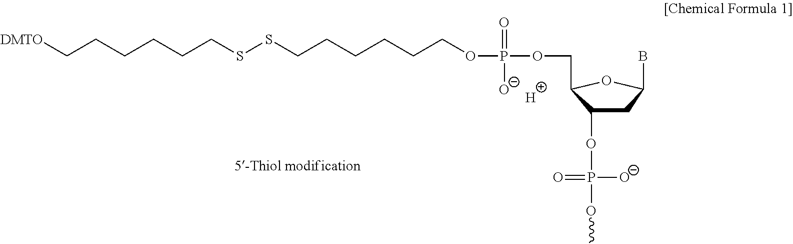

Specifically, the primer may be used by modifying the primer using a thiol modifier C6 S—S (O-Dimethoxytrityl-hexyl-dithiohexyl-phosphate), which is a compound having a thiol functional group, and more specifically, the primer may be used by further adding and attaching a spacer consisting of 5 to 30 adenine sequences A to the thiol modifier C6 S—S. The spacer may prevent spatial interference between neighboring primers when the primer attached to the surface of the magnetic nanoparticles performs the polymerase chain reaction, and improves the mobility of the primer to improve the efficiency of the polymerase chain reaction. On the other hand, the adenine sequences may be 5 to 30, and when the adenine sequences are less than 5, the function as the spacer is not sufficient, and when the adenine sequences are more than 30, the spacer is provided too long compared to the size of the magnetic nanoparticle to have a problem.

The primer may be mixed with the magnetic nanoparticles and bound to the surface of the magnetic nanoparticles. In this case, the salt aging may be performed in order to prevent non-specific attachment and agglomeration of the base sequence of the primer from occurring.

The salt aging may include adding and mixing the magnetic nanoparticles and the primers to a stirrer, and then adding a first solution and a second solution in a volume ratio of 5:1 to 30:1 in the stirrer a plurality of times and sonicating the mixture. The first solution may be provided by mixing a sodium chloride solution, a phosphate buffered saline, and ultrapure water, and the second solution may be prepared by mixing a phosphate buffered saline, sodium dodecyl sulfate and ultrapure water.

Specifically, the salt aging may include sequentially primary aging and secondary aging. The primary aging may be performed for 2 to 6 hours, and the sonication may be performed by adding the first and second solutions every 1 hour. The secondary aging may be performed for 4 to 12 hours, and the sonication may be performed by using 1.5 to 3-fold the first and second solutions used in the primary aging at a volume ratio and adding the first and second solutions every 2 hour interval. The salt aging is performed by the primary aging and the secondary aging by varying conditions to improve the process efficiency.

By applying the magnetic field under the first and second conditions, at least a part of the magnetic nanoparticle part may be transferred to the opening side of the reactor to be in contact with the conductive substrate. The first condition may include a condition of 0.1 T to 1 T for 1 minute to 10 minutes, and the second condition may include a condition of 0.1 T to 1 T for 12 minutes to 30 minutes.

The first condition is for primarily attaching the magnetic nanoparticle part to the conductive substrate, and the second condition is for adding metal ions including at least one of ruthenium (Ru), iron (Fe), silver (Ag), copper (Cu), nickel (Ni), cadmium (Cd), and zinc (Zn) and then moving the magnetic nanoparticle part to which the metal ions are adsorbed onto the conductive substrate. Alternatively, instead of the metal ions, methylene blue or a compound containing a nitro group may be added.

In the measuring of the electrochemical signal, the first electrode includes a reference electrode, the conductive substrate includes a working electrode, and the second electrode may include a counter electrode controlling an electron balance generated from the reference electrode and the working electrode, which are performed as a three-electrode module.

The first or second electrode may consist of any one or more of gold (Au), cobalt (Co), platinum (Pt), silver (Ag), carbon nanotube, graphene, and carbon, and the conductive substrate may include any one or more of indium tin oxide (ITO), ZnO, SnO₂, In₂O₃, CdSnO₄, a carbon substrate material including carbon nanotubes, a fluorine-doped tin oxide (FTO) added with fluorine, and an aluminum doped zinc oxide (AZO) added with aluminum.

The measuring of the electrochemical signal may include any one or more of a differential pulse voltammeter (DPV), an anodic stripping voltammetry (ASV), a chronoamperometry (CA), a cyclic voltammetry, a square wave voltammetry (SWV), and an impedance meter.

Specifically, the first electrode is a reference electrode containing Ag/AgCl, the second electrode is a counter electrode containing Pt, and the conductive substrate is a working electrode containing ITO, which are performed as the three-electrode module, and the measuring of the electrochemical signal may include a differential pulse voltammetry (DPV).

An initial current signal and a final current signal may be measured by measuring the electrochemical signal, and the initial current signal means a primary current signal, and the final current signal is higher than the initial current signal with little change in the current signal.

The higher the amplification density on the surface of the magnetic nanoparticle part and the greater the number of amplified magnetic nanoparticle parts, the greater the amount of adsorption with metal ions. For example, when the concentration of a magnetic nanoparticle part having a long base pair is low compared to a magnetic nanoparticle part attached with a short base sequence, a difference between the initial current amount and the maximum current increases. Specifically, the initial current signal is measured by applying a current in a form in which the magnetic nanoparticle part attached with the short base sequence and the magnetic nanoparticle part having the long base pair are arranged randomly, on the surface of the conductive substrate, the concentration of the magnetic nanoparticle part having the long base pair is low, so that the degree of the electric signal by the adsorbed metal ions is weak. On the other hand, as the current is repeatedly applied, the magnetic nanoparticle part having the long base pair is aligned adjacent to the surface of the conductive substrate, and the magnetic nanoparticle part attached with a relatively short base sequence is handed over to the magnetic nanoparticle part having the long base pair, and thus rearrangement to be far away from the conductive substrate may occur. As such current application is repeatedly performed, the current signal reaches the final current signal maintained in a somewhat constant state, and the target nucleic acids may be effectively detected by a difference between the initial current signal and the final current signal.

In the kit and the method for detecting the target nucleic acids using the magnetic nanoparticles according to an embodiment of the present disclosure, even when the concentration of the target nucleic acids in the sample is 1 zM or more, the target nucleic acids may be detected.

Hereinafter, Preparation Examples and Examples of the present disclosure will be described. However, the following Examples are only a preferred Example of the present disclosure, and the scope of the present disclosure is not limited by the following Examples.

Preparation Example 1 (Preparation of iron oxide nanoparticles) Iron oxide nanoparticles were synthesized through a polyol method. Iron chloride hexahydrate (FeCl₃·6H₂O) was used as an iron ion precursor, ethylene glycol serving as both a reducing agent and a solvent, sodium acetate (NaOAc) assisting hydrolysis, and H₂O were used, respectively. In a three-necked flask containing 50 mL of ethylene glycol, 2 mmol of FeCl₃·6H₂O, 6 mmol of NaOAc, and 150 mmol of H₂O were added, and rapidly heated to 200° C. for 15 minutes while mechanical stirring. The reaction time was maintained at 200° C. for 3 hours and 30 minutes, and then the mixture was cooled to room temperature and washed several times with ethanol to prepare iron oxide nanoparticles.

### Preparation Example 2 (Preparation of Gold Nanoparticles)

For the gold nanoparticles, gold chloride trihydrate (HAuCl₄.3H₂O) was used as a gold ion precursor, and sodium borohydride (NaBH₄) as a reducing agent, trisodium citrate as a stabilizer, and H₂O as a solvent were used for the reaction, respectively. First, the concentration was adjusted with 0.25 mM HAuCl₄.3H₂O and 0.25 mM trisodium citrate in 50 mL of H₂O, and then 1.5 mL of cold H₂O (NaBH₄, 0.1 M) was added. In this case, while the temperature was kept at room temperature, the magnetic stirring reaction was performed using a magnetic bar.

Preparation Example 3 (preparation of magnetic nanoparticles) By using the iron oxide nanoparticles prepared in Preparation Example 1, silica was formed on the surface of the iron oxide nanoparticles using a Stφber method using a TEOS hydrolysis reaction under an ammonia catalyst. Ethanol and H₂O were used as solvents, and polyvinylpyrrolidone (PVP) as a stabilizer, ammonium hydroxide solution (NH₄OH) as a catalyst, and tetraethyl orthosilicate (TEOS) as a silica precursor were used, respectively. In the reactor, 50 mL of ethanol, 7.5 mL of H₂O, 2.5 mL of NH₄OH, and 400 mg of PVP were added and mixed. Then, 25 mg of the iron oxide nanoparticles prepared in Preparation Example 1 were added. After 0.050 mL of TEOS was added to a uniform mixed solution mixed with the iron oxide nanoparticles, shaking was performed at room temperature for 1 hour and 30 minutes. After the reaction was completed, the prepared pre-magnetic nanoparticles (magnetic nanoparticles with silica formed on the surface) were dispersed in 10 mL of ethanol after washing several times with ethanol.

In order to attach the gold nanoparticles prepared in Preparation Example 2 to the pre-magnetic nanoparticles, 3-aminopropyl triethoxysilane (APTES) was used as a precursor of an amine group (—NH₂), and 2-propanol was used as a solvent. 4 mL of ethanol in which pre-magnetic nanoparticles were dispersed was replaced with a solvent of 10 mL of 2-propanol, and 0.050 mL of APTES was added to perform shaking. Then, the reaction was performed by sonication at 80° C. for 4 hours. After the reaction was completed, the prepared pre-magnetic nanoparticles functionalized with the amine group on the surface were washed with H₂O and dispersed in 5 mL of H₂O. 5 mL of H₂O in which the pre-magnetic particles functionalized with an amine group on the surface were dispersed and 30 mL of gold nanoparticles were mixed together, and then shaken for 16 hours. Subsequently, H₂O dissolved in 1 wt % of PVP was uniformly shaken with a stabilizer to prepare pre-magnetic nanoparticles with gold nanoparticles attached to the surface, washed with H₂O, and dispersed in 20 mL of H₂O. Here, the gold nanoparticles are bound to the pre-magnetic nanoparticles through the amine group.

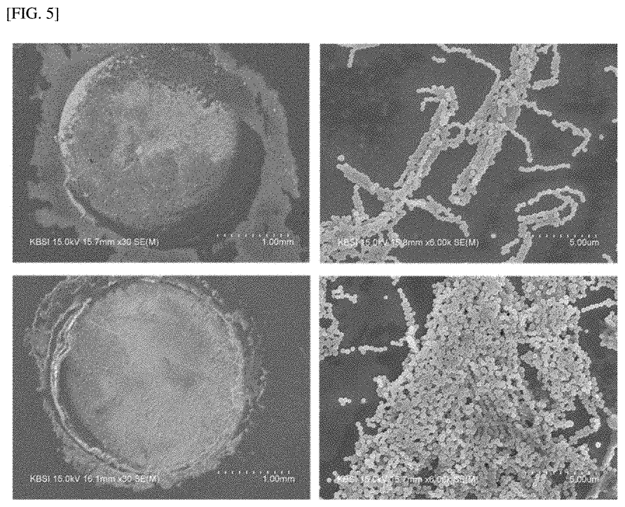

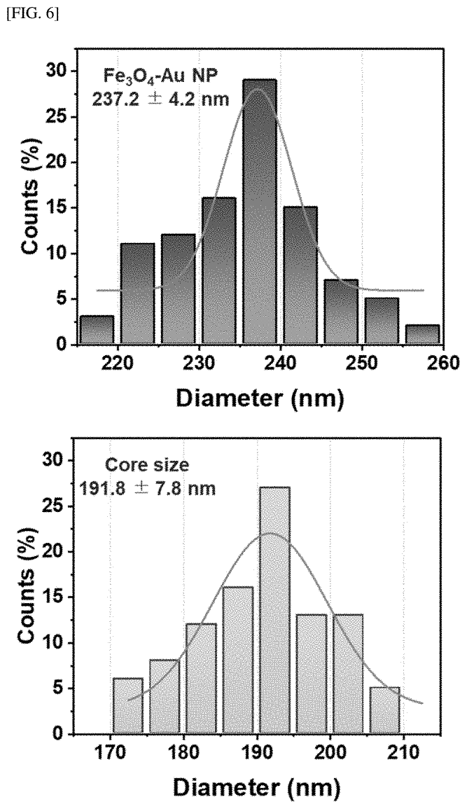

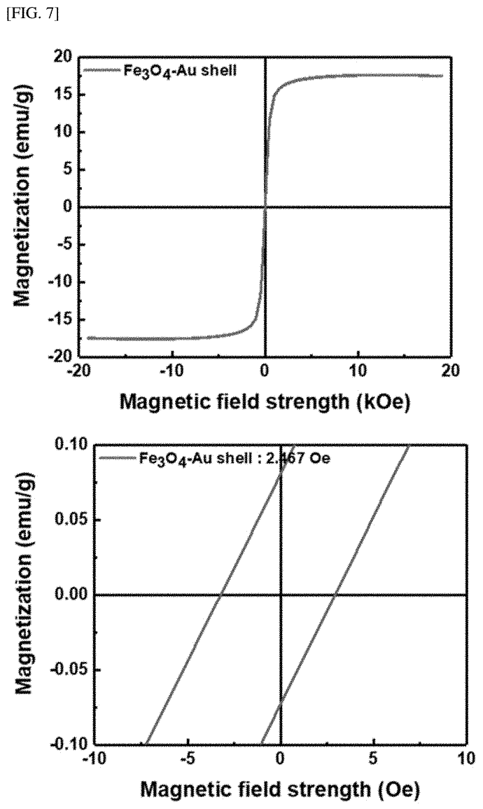

For a gold nanoparticle growth solution, 45 mg of HAuCl₄.3H₂O and 50 mg of potassium carbonate (K₂CO₃) were dissolved in 500 mL of water, aged for 16 hours, and then used as the gold nanoparticle growth solution. 1 mg of the pre-magnetic particles functionalized on the surface with the amine group was dispersed in 2 mL of 1 wt % of polyninylpyrrolidone (PVP, 55,000 kDa), and then mixed with 80 mL of the gold nanoparticle growth solution and 1 mg/mL 3 mL of bis(p-sulfonatophenyl)phenylphosphine dihydrate dipotassium salt (BSPP). Subsequently, 2 mL of 37 wt % formaldehyde was injected and shaken for 2 hours to prepare magnetic nanoparticles having a cord part made of iron oxide particles and a shell portion made of gold. The prepared magnetic nanoparticles were washed with H₂O and dispersed in 20 mL of H₂O. FIG. 4 is a schematic diagram and a TEM image of magnetic nanoparticles of Preparation Example 3. FIG. 5 is an SEM image of the magnetic nanoparticles of Preparation Example 3. FIG. 6 is a graph showing sizes of the magnetic nanoparticles of Preparation Example 3 and the iron oxide nanoparticles (core) according to Preparation Example 1. FIG. 7 is a result of confirming magnetic field characteristics of the magnetic nanoparticles of Preparation Example 3.

Referring to FIGS. 4 to 6, it was confirmed that the diameter of the iron oxide nanoparticles was about 191.8 nm, and the diameter of the magnetic nanoparticles with gold on the surface was about 237 nm, and the particle size was also formed evenly. Referring to FIG. 7, it was confirmed that the eminence when a field is 0 indicates 0, and the coercivity is measured as small as 0 so that agglomeration does not occur because it is required to be amplified. In addition, it was confirmed that a maximum magnetic field exhibited a high value of about 16.

### Preparation Example 4 (Preparation of Magnetic Nanoparticles Bound with Reverse Primer)

In a 5′ direction of a reverse primer capable of reacting with the 5′ vicinity of a specific single base sequence, a double thiol group is bound, so that a double thiol bond in the 5′ direction of the reverse primer was first processed into a single thiol bond for attachment to the surface of the magnetic nanoparticles.

2 μL of 2 M NaOH was added to 5 mL of phosphate-buffered saline (PBS, 100 mM, pH 7.4) to control pH to 8, which does not affect the base sequence. Here, 0.10283 g of 1,4-dithiothreitol was added and mechanically stirred for 1 minute to prepare a DTT solution. In order to process the base sequence of the reverse primer with a single thiol bond, 75 μL of a 100 μM reverse primer solution and 25 μL of the DTT solution prepared above were injected into a micro tube and mechanically stirred for 1 hour to prepare a primer mixture.

A NAP-5 column was prepared and 10 mL of ultra purified water (UH₂O) was sequentially injected into the NAP-5 column by 3 mL, 3 mL, 3 mL, and 1 mL to perform stabilization (equilibration), respectively. When the primer mixture prepared by mechanical stirring for 1 hour was injected into the NAP-5 column and the primer mixture was completely permeated from the top of the column, 500 μL of UH₂O flowed into the column. After 500 μL of UH₂O fully passed through the NAP-5 column, a microtube was placed at the bottom of the NAP-5 column. Then, while additionally injecting 500 L of UH₂O, 500 μL of the resultant was stored in the microtube until completely flowing out.

UH₂O was quantitatively prepared in 250 μL. 10 μg of magnetic nanoparticles was dispersed in 250 μL of the prepared UH₂O, centrifuged at 13500 rpm for 7 minutes, and then washed 3 times. 500 μL of a single thiolated reverse primer finally purified using a NAP-5 column and 250 μL of the magnetic nanoparticles washed after centrifugation were mixed and mounted on a shaker, and then shaken overnight. Salt-aging was performed to eliminate non-specific attachment and agglomeration of the base sequence of the reverse primer. A salt-aging solution was prepared as a mixed solution of 2 mL of 5 M NaCl, 0.5 mL of 100 mM PBS, and 2.5 mL of UH₂O, and a PB/SDS solution was prepared as a mixed solution of 500 μL of 0.1 M PBS, 10 mg of sodium dodecyl sulfate (SDS), and 9.5 mL of UH₂O, respectively. Immediately after mixing the single thiolated reverse primer and the magnetic nanoparticles, 1.58 μL of the PB/SDS solution and 14.24 μL of the salt-aging solution were added every hour 4 times for 3 hours and then sonicated. From 4 hours to 8 hours after mixing the single thiolated reverse primer and the magnetic nanoparticles, 3.16 μL of the PB/SDS solution and 28.49 L of the salt-aging solution were added every 2 hours, respectively, and then sonicated.

After reacting overnight, the mixture was washed at least 3 times, and after washing was completed, 5 μL of the PBS solution was injected so that the amount of the magnetic nanoparticles bound to the reverse primer was 5 μL.

### Example 1

1. Preparation of Magnetic Nanoparticles with Amplified Product on Surface (Breast Cancer)

5 μL of the magnetic nanoparticles attached with the reverse primer, 1 μL of a forward primer single sequence, 2 μL of a target single sequence as target nucleic acids, 10 μL of power mix, and 2 μL of UH₂O were injected into a PCR tube. Sonication was performed for 1 minute so that all the injected solutions were uniformly mixed. After repeating polymerase chain reaction (PCR) 40 times, the magnetic nanoparticles to which the amplified product was finally attached were washed 3 times and then dispersed in 5 L of UH₂O.

Here, the setting of the PCR may vary depending on the sequence and size of a target DNA. In the case of an estrogen receptor alpha (ESRα), a specific biomarker for breast cancer, a target single sequence is 5′-AGTCCTTTCTGTGTCTTCCCACCTACAGTAACAAAGGCATGGAGCATCTGTA CAGCATGAAGTGCAAGAACGTGGTGCCCCTCTATGACCTGCTGCTGGAGAT GCTGGA-3′ consisting of 109 mer. The reverse primer used a modified reverse primer of 5′ thiol C6 S-S-AAAAAAAAAAAAAAATCCAGCATCTCCAGCAGCA-3′ with a thiol group attached to the end and additionally attached with sequence A 15 mer for inhibiting non-specific binding. The forward primer that reacts with a complementary single sequence of the target single sequence in the aqueous solution during the PCR was composed of 5′-AGTCCTTTCTTGTGTCTTCCCA-3′. Here, the thiol group attached to the reverse primer was modified with the thiol modifier C6 S-S (O-Dimethoxytrityl-hexyl-dithiohexyl-phosphate).

2. Evaluation of Detection of Target Nucleic Acids Using Magnetic Nanoparticles

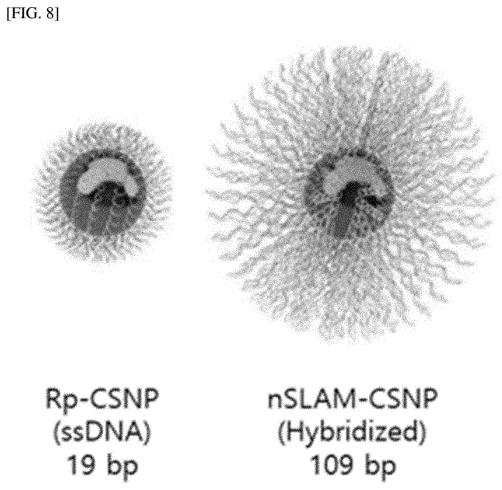

FIG. 8 is a diagram schematically illustrating a comparison before and after performing polymerase chain reaction (PCR). When the target base sequence of the target nucleic acids and the reverse primer for specific amplification are attached to magnetic nanoparticles (iron oxide (core portion)—gold (shell portion)), that is, when there is a specific disease, after the PCR, the base sequence is amplified in a double helix structure as much as the length of the target on the surface of the magnetic nanoparticles. Compared to the magnetic nanoparticles to which only the reverse primer was attached before performing the PCR, magnetic nanoparticles having the amplified product after performing the PCR are longer than 80 mer in the case of ESRα in the attachment direction of DNA, and then the shape itself is physically formed as a giant product to prepare 5 μL of the magnetic nanoparticles with the amplified product.

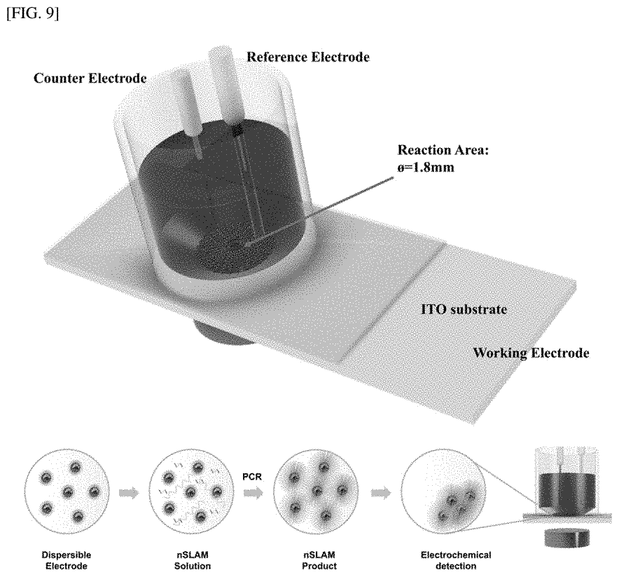

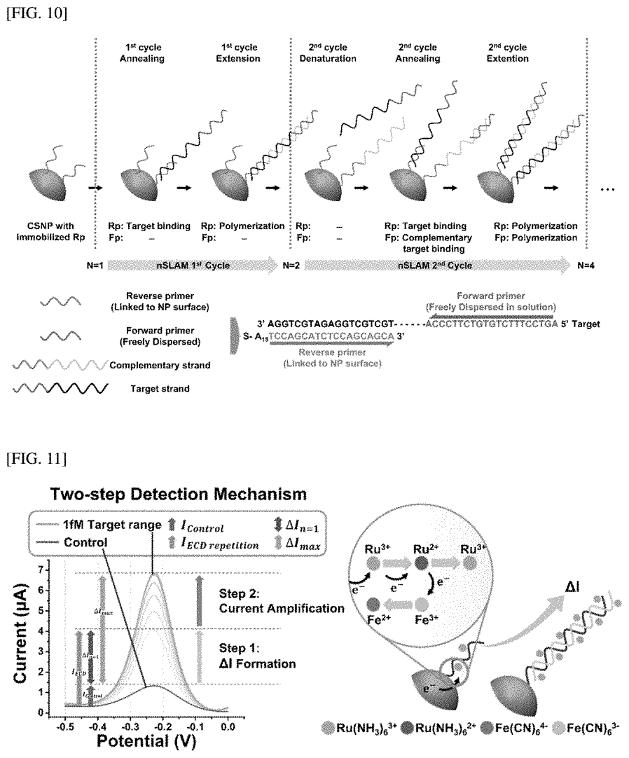

FIG. 9 is a diagram schematically illustrating a device for detecting target nucleic acids using magnetic nanoparticles of the present disclosure and a method of using the same. FIG. 10 is a diagram schematically illustrating an amplification reaction mechanism using the magnetic nanoparticle part in FIG. 9. FIG. 11 is a schematic diagram illustrating a difference in current signal between a magnetic nanoparticle having a product with amplified long base pairs and a magnetic nanoparticle having only a reverse primer.

The method was performed using the device illustrated in FIG. 9, and an electrochemical detection container having a circular opening (with a radius of 1.8 mm) at the bottom was used, and an opening was closely adhered to the electrochemical detection container to prevent leakage using an ITO substrate. The ITO substrate was used with 10 Ω/sq/20 mm×30 mm/0.7 T and washed with acetone, ethanol, and water three times before adhering to the electrochemical detection container. 5 μL of the amplified product (including magnetic nanoparticles) in which the PCR has been completed was injected into the circular opening at the bottom of the electrochemical detection container and placed in contact with the ITO substrate. In this case, the magnetic nanoparticles included in the amplified product are in a state where the magnetic nanoparticles having the amplified product and the magnetic nanoparticles having only the reverse primer are mixed according to a conditions and the number of times of the PCR, and a concentration of the target nucleic acids. Here, after a magnetic field (about 3000 to 5000 Gauss with a neodymium magnet) was applied for 5 minutes and then maintained so that all the magnetic nanoparticles included in 5 μL of the amplified product may be attached to the ITO substrate, the applied magnetic field was slowly removed. Subsequently, 1 mL of a solution in which 50 μM Ru(NH₃)₆Cl and 4 mM K₃Fe(CN)₆ were mixed in PBS was injected into the electrochemical detection container, and then a magnetic field was further applied for 15 minutes, so that the magnetic nanoparticles are maintained in contact with the ITO substrate in the circular opening at the bottom of the electrochemical detection container.

After 15 minutes, using a potentiostat (EM-stat3/Palmsense Co., Ltd.), a tube-type Ag/AgCl reference electrode was bonded to a reference electrode, a post-type Pt counter electrode was bonded to the counter electrode, and a working electrode was bonded to the ITO substrate. After the three-electrode setting was completed, the DPV mode measurement was performed in an EM-stat3 device. In this case, set values of a pulse height 50 mV, a pulse width 0.05 s, a step height 5 mV, and a step width 0.1 s were input and a voltage of 0 to −0.5V was applied to be measured.

Referring to FIG. 10, in the magnetic nanoparticles having the reverse primer attached to the surface, in the process of amplifying the reverse primer in the target single sequence, a forward primer was amplified on the surface of the magnetic nanoparticles, and finally amplified on the surface of the magnetic nanoparticles rather than in the aqueous solution. During the PCR, the reverse primer on the surface of the magnetic nanoparticles first reacted with the target DNA, which was the target nucleic acids to amplify a single base sequence. Thereafter, the forward primer second reacted with the reverse primer amplified on the surface of the magnetic nanoparticles to generate a single long double-helix base sequence pair on the surface of the magnetic nanoparticles. In this case, the generated long double helix was a target base sequence and a complementary sequence to the target base sequence. This reaction occurred every cycle of the PCR, and A(2ⁿ−1) [A=initial concentration of the target base sequence, n=the cycle number of PCR] were collected on the surface of the magnetic nanoparticles. That is, there was a difference between the number of magnetic nanoparticles having the amplified product on the surface and the amplification degree on the surface in the case of the magnetic nanoparticles having the amplified product according to the concentration of the target single sequence during the PCR and the cycle number of the PCR.

Based on the above-mentioned variables, there was a difference even in the state of the magnetic nanoparticles having the final amplified product after the completion of the PCR. In FIG. 11, the concentration of the target nucleic acids was confirmed as 1 fM. Here, it may be described by the two-step detection mechanism, and compared to the initial current signal by repeated DPV measurement, each current signal gradually increases, and the increase in the current signal has the same meaning as the increase in sensitivity. In the case of measuring the DPV using only the conventional substrate, since the substrate itself is of a fixed type, the current signal hardly changes even if the DPV measurement is performed several times. On the other hand, in the case of the embodiment, in the case of repetitively measuring the DPV, since the surface condition of the magnetic nanoparticles is different, magnetic nanoparticles to which a longer DNA sequence is attached tend to be closer to the ITO substrate than magnetic nanoparticles to which a short DNA sequence is attached, which contributes to an increase in the current signal, and current amplification occurs. That is, in a conventional art, it is determined as a signal corresponding to a first current signal generated in the present disclosure, whereas in the present disclosure, it can be determined based on an amplified current signal rather than the first current signal, so that it is possible to be determined with high sensitivity.

That is, when the amplification density is high and the number of magnetic nanoparticles having the amplified product on the surface is large, the relative length of the DNA sequence is also increased. In addition, when the amplification is completed, since the magnetic nanoparticles having the amplified product has a double helix structure rather than a single sequence, a difference occurs in the degree of adsorption of Ru³⁺ ions compared to the magnetic nanoparticles having only the reverse primer, which is a short single sequence. Since the base sequence is negatively charged, the Ru³⁺ ions are adsorbed to the base sequence, and thus, the degree of adsorption of Ru³⁺ ions is increased in the magnetic nanoparticles having the amplified product compared to the magnetic nanoparticles having only the reverse primer. In addition, even among the magnetic nanoparticles having the amplified product, the Ru³⁺ ions are more adsorbed as the length of the base sequence is increased and the base pair rather than a single sequence is formed. When the concentration of the magnetic nanoparticles having a long base pair is lower than the concentration of the magnetic nanoparticles attached with a short base sequence, there is a difference in the maximum current signal compared to the initial current signal. The initial current signal measures the current signal of magnetic nanoparticles arranged randomly, and in this case, since the number of magnetic nanoparticles having the amplified product of the long base pair acting directly on the ITO substrate is small when the number of magnetic nanoparticles amplified with a relatively long base pairs is small, the degree of an electrical signal by the adsorbed Ru³⁺ ions is also weak (initial current signal).

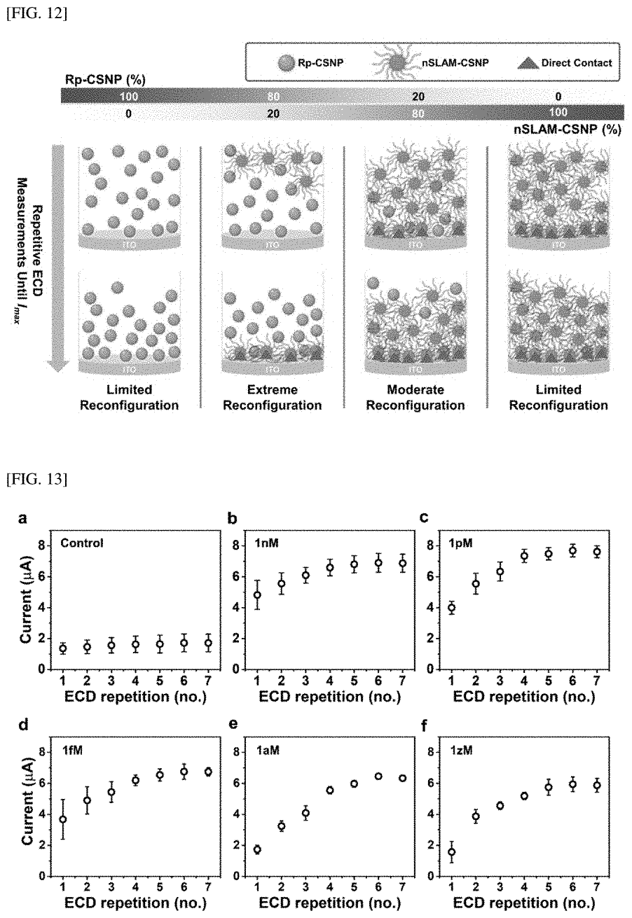

FIG. 12 is a schematic description of a rearrangement of magnetic nanoparticles that cause a difference between an initial current signal and a maximum current signal.

When the current is applied while repeatedly performing the DPV measurement, the magnetic nanoparticles having the amplified product of the long base pair are attracted to the ITO substrate, and as a result, the magnetic nanoparticles are realigned on the ITO substrate. That is, as the DPV measurement is repeatedly performed, the number of magnetic nanoparticles having the amplified product in direct contact with the ITO substrate increases, and the magnetic nanoparticles having only the reverse primer are relatively far away from the ITO substrate.

As a result, when the DPV measurement is sufficiently performed, the magnetic nanoparticles having the amplified product of the long base pair are maximally collected on the ITO substrate, and accordingly, the current signal has a larger value than the initial current signal. This phenomenon appears larger as the number of magnetic nanoparticles having the amplified product of the long base pair decreases, and it is determined that as the cycle number of the PCR decreases and the concentration of the initial target base sequence decreases, the proportion of the magnetic nanoparticles having only the reverse primer is more increased than the magnetic nanoparticles having the amplified product of the long base pair in the final product of the PCR. That is, the difference between the initial current signal and the maximum current signal is caused by rearrangement between the magnetic nanoparticles having the amplified product of the long base pair in contact with the ITO substrate and the magnetic nanoparticles having only the reverse primer.

FIG. 13 is a result of confirming current signals according to the number of DPV measurements by varying a concentration of target nucleic acids using a magnetic nanoparticle (control) attached with only a reverse primer and a magnetic nanoparticle having an amplified product, respectively. In FIG. 13A, in the case of the magnetic nanoparticles attached with only the reverse primer, it was confirmed that there was no change in the height of the current even when the DPV was applied. On the other hand, referring to FIGS. 13B to 13F, it was confirmed that as the DPV is repeatedly measured up to 7 times, the height of the current gradually increases, and the maximum value of the current depends on the concentration of the target nucleic acids. It can be seen that the maximum current value is shown at DPV 6 times.

In FIG. 13B, as the result of performing the PCR at a concentration of 1 nM of the target nucleic acids and detecting the electrochemical signal, it was confirmed that the repeated measurement result exhibited a current height of 4 μA or more from a first signal. In FIGS. 13B to 13F, as the result of repetitively measuring the DPV for the concentrations of the initial target base sequence of 1 nM, 1 pM, 1 fM, 1 aM, and 1 zM, respectively, it was confirmed that as the concentration of target nucleic acids was decreased, the current height of the DPV repetitively measured up to 7 times as well as once signal was entirely lowered.

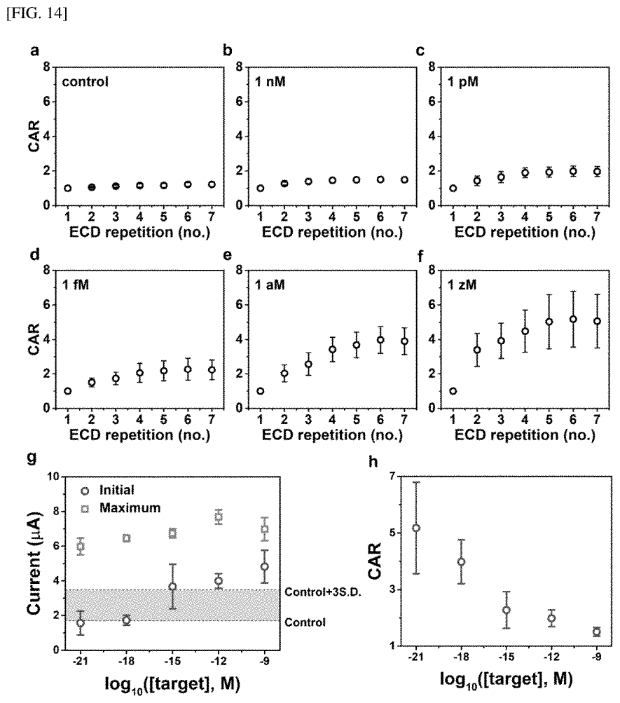

FIG. 14 illustrates the results of FIG. 13 by a current amplified ratio (CAR). Here, a CAR index [CAR=current after repeated measurement/initial current] does not simply indicate the height of the current, but indicates the degree to which the current signal rises compared to the initial current signal b repeated performing the DPV. It was confirmed that when the concentration of target nucleic acids was low, that is, when the amplified product was small, the CAR index increased significantly. Referring to FIG. 14H, the CAR in all concentration areas was confirmed, and it was equally confirmed that when the amount of the amplified product was small, the CAR index increased with a larger width. In FIG. 14G, as a result of indicating the result of measuring the current height from 1 nM to 1 zM as one index, compared to magnetic nanoparticles (control) attached with only a reverse primer, the current is significantly increased in all of the concentration areas. Since the initial current signal measured in the concentration area of 1 fM or less is included in the range of control+3 S.D., it is not easy to reliably determine the presence or absence of disease. It is possible to improve the electrochemical detection sensitivity compared to the initial current signal through the maximum current signal, which is the signal amplified by repeated DPV measurement, and as a result, it is possible to determine the presence or absence of disease detection in the concentration area which has been hardly determined previously.

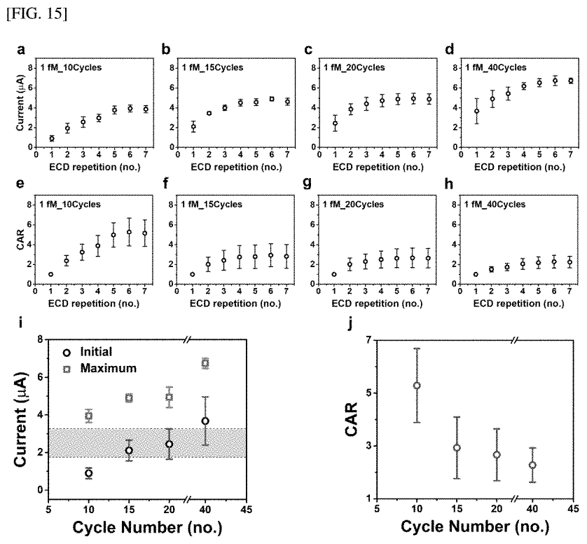

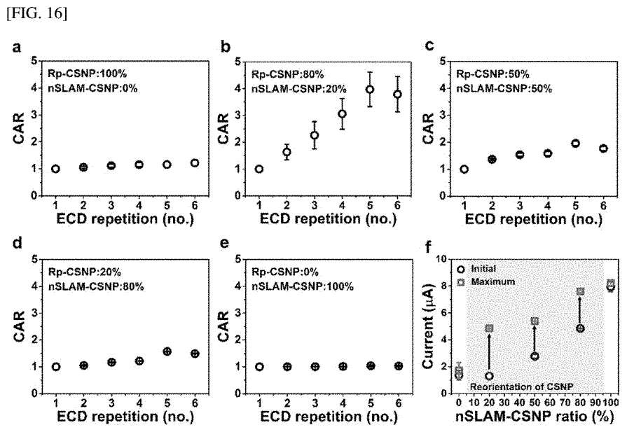

FIG. 15 is a result of confirming magnetic nanoparticles (control) attached with only a reverse primer and magnetic nanoparticles having an amplified product by reducing the cycle number of polymerase chain reaction. FIG. 15 illustrates results of setting the concentration of target nucleic acids to 1 fM and reducing the cycle number of the PCR from previous 40 times to 20 times, 15 times, and 10 times, respectively.

FIGS. 15A and 15E illustrate a current height and a CAR index when measuring DPV as a result of 10 times PCR in a 1 fM area, FIGS. 15B and 15F illustrate a current height and a CAR index when measuring DPV as a result of 15 times PCR in the 1 fM area, FIGS. 15C and 15G illustrate a current height and a CAR index when measuring DPV as a result of 20 times PCR in the 1 fM area, and FIGS. 15D and 15H illustrate a current height and a CAR index when measuring DPV as a result of 40 times PCR in the 1 fM area.

Referring to FIG. 15(i), as a result of measuring the DPV according to the cycle number of PCR of 40 times, 20 times, 15 times, and 10 times in the 1 fM area, the result was compared with that of control+3 S. D. When 10 cycles were performed, it was found that the maximum current value was adjacent to control+3 S. D. In the present disclosure, it was confirmed that when the concentration of the target nucleic acids was 1 fM, the determination was possible when only 10 cycles of the PCR were performed. FIG. 16 is a result confirmed by varying a proportion of magnetic nanoparticles (control) attached with only a reverse primer and magnetic nanoparticles having an amplified product. Referring to FIG. 16, in the case of repeated DPV measurement, while the magnetic nanoparticles are rearranged on the ITO substrate, the current signal gradually increases compared to the initial current signal to exhibit the maximum current signal. With respect to the magnetic nanoparticles attached with only the reverse primer, the proportion of the magnetic nanoparticles having the amplified product was increased to 0%, 23%, 50%, 80%, and 100%, respectively. It was confirmed that the increase in the current signal was greatest in the case of magnetic nanoparticles having 20% of the amplified product, and the current signal was decreased in the case of 100%. That is, it was confirmed that while the DPV was repeatedly performed, the rearrangement of the magnetic nanoparticles occurred, which affected a change in the current signal.

Those skilled in the art will be able to understand that the present disclosure can be easily executed in other detailed forms without changing the technical spirit or an essential feature thereof. Therefore, it should be appreciated that the embodiments described above are illustrative in all aspects and are not restricted. The scope of the present disclosure is represented by claims to be described below rather than the detailed description, and it is to be interpreted that the meaning and scope of the claims and all the changes or modified forms derived from the equivalents thereof come within the scope of the present disclosure.

What is claimed is:

1. A kit for detecting target nucleic acids using magnetic nanoparticles comprising:
a reactor having an opening on one side;
at least one magnetic nanoparticle part provided in the reactor;
a conductive substrate provided to cover the opening of the reactor; and
a magnetic field applying part for applying a magnetic field to the reactor,
wherein the magnetic nanoparticle part includes a magnetic nanoparticle including a core portion made of iron oxide and a shell portion made of gold and provided to surround the core portion, and a primer attached to the shell portion of the magnetic nanoparticle.

2. The kit for detecting target nucleic acids using magnetic nanoparticles of claim 1, wherein an average diameter of the magnetic nanoparticle is provided with a first length and an average thickness of the shell portion is provided with a second length, wherein the second length is 0.09 to 0.15 times greater than the first length.

3. The kit for detecting target nucleic acids using magnetic nanoparticles of claim 1, wherein an average diameter of the magnetic nanoparticle is provided with a first length and an average thickness of the shell portion is provided with a second length, wherein the first length is 170 nm to 300 nm and the second length is 15 nm to 40 nm.

4. The kit for detecting target nucleic acids using magnetic nanoparticles of claim 1, wherein the magnetic nanoparticle part is prepared by preparing a core portion made of iron oxide,
providing a buffer portion containing silicon on the surface of the core portion,
preparing magnetic nanoparticles by functionalizing an outer surface of the buffer portion with at least one of an amino group (—NH₂) and a thiol group (—SH) and forming a shell portion containing gold thereon, and
mixing the magnetic nanoparticles and a thiolated reverse primer.

5. The kit for detecting target nucleic acids using magnetic nanoparticles of claim 1, wherein the primer includes a reverse primer, and
further comprising an electrochemical signal measuring part for measuring an electrochemical signal using a first electrode, a second electrode, and a three-electrode module including the first electrode, the second electrode, and a conductive substrate, which are provided in the reactor.

6. The kit for detecting target nucleic acids using magnetic nanoparticles of claim 5, wherein the first electrode includes a reference electrode, the conductive substrate includes a working electrode, and the second electrode includes a counter electrode controlling an electron balance generated from the reference electrode and the working electrode, which are performed by a three-electrode module,
the first or second electrode consists of any one or more of gold (Au), cobalt (Co), platinum (Pt), silver (Ag), carbon nanotube, graphene, and carbon, and
the conductive substrate includes any one or more of indium tin oxide (ITO), ZnO, SnO₂, In₂O₃, CdSnO₄, a carbon substrate material including carbon nanotubes, a fluorine-doped tin oxide (FTO) added with fluorine, and an aluminum doped zinc oxide (AZO) added with aluminum.

7. The kit for detecting target nucleic acids using magnetic nanoparticles of claim 6, wherein the electrochemical signal measuring part includes any one or more of a differential pulse voltammeter (DPV), an anodic stripping voltammetry (ASV), a chronoamperometry (CA), a cyclic voltammetry, a square wave voltammetry (SWV), and an impedance meter.

8. The kit for detecting target nucleic acids using magnetic nanoparticles of claim 1, wherein the magnetic field applying part reversibly applies a magnetic field into the reactor one or more times with the conductive substrate interposed therebetween, and
the magnetic field applying part moves the magnetic nanoparticle part toward the opening of the reactor and controls the degree of moving the magnetic nanoparticle part.

9. The kit for detecting target nucleic acids using magnetic nanoparticles of claim 1, wherein a sample containing the target nucleic acids includes any one or more of blood, serum, plasma, saliva, ascites, amniotic fluid, semen, lacrimal fluid, cerebrospinal fluid, bone marrow, pleural fluid, synovial fluid, lymph, urine, tissue biopsies and cell lines.

10. The kit for detecting target nucleic acids using magnetic nanoparticles of claim 1, wherein the target nucleic acid is cell free DNA (cfDNA) or circulating tumor DNA (ctDNA).

11. A method for detecting target nucleic acids using magnetic nanoparticles comprising the steps of:
preparing a magnetic nanoparticle part;
obtaining a magnetic nanoparticle part with an amplified product collected on the surface by adding the magnetic nanoparticle part in a sample including target nucleic acids to perform polymerase chain reaction (PCR);
adding the magnetic nanoparticle part with the amplified product collected on the surface in the reactor provided with an opening, covering the opening with a conductive substrate, and applying a magnetic field into the reaction from one side of the conductive substrate under a first condition;
adding metal ions including any one or more of ruthenium (Ru), iron (Fe), silver (Ag), copper (Cu), nickel (Ni), cadmium (Cd) and zinc (Zn) into the reactor and then applying the magnetic filed under a second condition; and
providing first and second electrodes in the reactor, and measuring an electrochemical signal using the first and second electrodes and the conductive substrate together.

12. The method for detecting target nucleic acids using magnetic nanoparticles of claim 11, wherein the preparing of the magnetic nanoparticle part comprises
preparing a core portion made of iron oxide,
providing a buffer portion including silicon on the surface of the core portion to prepare a pre-nanoparticle,
preparing a magnetic nanoparticle by functionalizing an outer surface of the buffer portion with at least one of an amino group (—NH₂) and a thiol group (—SH) and forming a shell portion containing gold thereon, and
mixing the magnetic nanoparticle and a primer and performing salt aging.

13. The method for detecting target nucleic acids using magnetic nanoparticles of claim 12, wherein an average diameter of the magnetic nanoparticle is provided with a first length and an average thickness of the shell portion is provided with a second length, wherein the first length is 170 nm to 300 nm and the second length is 15 nm to 40 nm.

14. The method for detecting target nucleic acids using magnetic nanoparticles of claim 12, wherein the preparing of the magnetic nanoparticle comprises
impregnating the pre-nanoparticles in a solution containing an amine group and then sonicating the pre-nanoparticles at a temperature of 60° C. to 90° C. for 2 hours to 10 hours,
washing the pre-nanoparticles using water after the sonication is completed and dispersing the washed pre-nanoparticles in water to prepare a pre-nanoparticle dispersion, and
forming a shell portion by mixing gold nanoparticles and then adding a chloroauric acid (HAuCl₄) solution to prepare the pre-nanoparticle dispersion as magnetic nanoparticles,
wherein the gold nanoparticles are prepared in advance in the form of particles having an average diameter of 0.5 nm to 5 nm and used as a seed.

15. The method for detecting target nucleic acids using magnetic nanoparticles of claim 12, wherein the primer includes a thiolated primer, and
the salt aging includes adding and mixing the magnetic nanoparticles and the primers to a stirrer, and then adding a first solution and a second solution in a volume ratio of 5:1 to 30:1 in the stirrer a plurality of times and sonicating the mixture,
wherein the first solution is provided by mixing a sodium chloride solution, a phosphate buffered saline, and ultrapure water, and the second solution is provided by mixing a phosphate buffered saline, sodium dodecyl sulfate and ultrapure water.

16. The method for detecting target nucleic acids using magnetic nanoparticles of claim 11, wherein by applying the magnetic field under the first and second conditions, at least a part of the magnetic nanoparticle part is transferred toward the opening of the reactor to be in contact with the conductive substrate,
the first condition includes a condition of 0.1 T to 1 T for 1 minute to 10 minutes, and
the second condition includes a condition of 0.1 T to 1 T for 12 minutes to 30 minutes.

17. The method for detecting target nucleic acids using magnetic nanoparticles of claim 11, wherein in the measuring of the electrochemical signal,
the first electrode includes a reference electrode, the conductive substrate includes a working electrode, and the second electrode includes a counter electrode controlling an electron balance generated from the reference electrode and the working electrode, which are performed as a three-electrode module,
the first or second electrode consists of any one or more of gold (Au), cobalt (Co), platinum (Pt), silver (Ag), carbon nanotube, graphene, and carbon, and
the conductive substrate includes any one or more of indium tin oxide (ITO), ZnO, SnO₂, In₂O₃, CdSnO₄, a carbon substrate material including carbon nanotubes, a fluorine-doped tin oxide (FTO) added with fluorine, and an aluminum doped zinc oxide (AZO) added with aluminum.

18. The method for detecting target nucleic acids using magnetic nanoparticles of claim 17, wherein the measuring of the electrochemical signal includes any one or more of a differential pulse voltammeter (DPV), an anodic stripping voltammetry (ASV), a chronoamperometry (CA), a cyclic voltammetry, a square wave voltammetry (SWV), and an impedance meter.

19. The method for detecting target nucleic acids using magnetic nanoparticles of claim 18, wherein the first electrode is a reference electrode containing Ag/AgCl, the second electrode is a counter electrode containing Pt, and the conductive substrate is a working electrode containing ITO, which are performed as a three-electrode module, and
the measuring of the electrochemical signal includes a differential pulse voltammetry (DPV).
